(19) United States
(12) Patent Application Publication
(10) Pub. No.: US 2025/0160938 A1
(43) Pub. Date: May 22, 2025

(54) CATHETER INCLUDING CONDUCTIVE FILARS

(71) Applicant: Medtronic Ireland Manufacturing Unlimited Company, Dublin (IE)

(72) Inventor: Cian Walsh, Galway (IE)

(21) Appl. No.: 18/839,619

(22) PCT Filed: Feb. 22, 2023
(86) PCT No.: PCT/EP2023/054432

§ 371 Date: Aug. 19, 2024

Related U.S. Application Data
(60) Provisional application No. 63/268,426, filed on Feb. 23, 2022.

(51) Int. Cl. A61B 18/14; A61B 17/00; A61B 18/00; B23K 26/362

(52) U.S. Cl. CPC A61B 18/1492; B23K 26/362; A61B 2017/00526; A61B 2017/00867; A61B 2018/00077; A61B 2018/00083; A61B 2018/00404; A61B 2018/00434; A61B 2018/00511; A61B 2018/00577; A61B 2018/1467

(57) ABSTRACT

A catheter that includes at least one therapeutic element and a plurality of electrically conductive filars configured to both electrically connect the at least one therapeutic element to a medical device. and to enable a portion of the catheter to expand radially outwards from a relatively low profile delivery configuration to a deployed configuration. Each filar of the plurality of filars includes an outer layer formed from a first material and an inner layer (also referred to herein as an inner core) radially inward of the outer layer and formed from a second material that is different from the first material. One of the first material or the second material comprises an electrically conductive material and the other of the first material or the second material comprises a shape memory material.

### TECHNICAL FIELD

The present technology is related to catheters.

### BACKGROUND

Catheters including one or more energy delivery elements have been proposed for use in various medical procedures, including neuromodulation procedures. For example, some catheters include a plurality of electrodes configured to deliver radiofrequency energy to a region of tissue during an ablation procedure.

### SUMMARY

The present disclosure describes catheters that include at least one therapeutic element and a plurality of electrically conductive filars configured to both electrically connect the at least one therapeutic element to a medical device, e.g., a source of radiofrequency energy, and to enable a portion of the respective catheter to expand radially outwards from a relatively low profile delivery configuration to a deployed configuration. The plurality of conductive filars can be arranged, for example, to define a tubular body that forms part of an elongated body of the catheter. Each filar of the plurality of filars includes an outer layer formed from a first material and an inner layer (also referred to herein as an inner core) radially inward of the outer layer and formed from a second material that is different from the first material. One of the first material or the second material comprises an electrically conductive material and the other of the first material or the second material comprises a shape memory material. The electrically conductive material enables the filars to electrically connect the at least one therapeutic element (e.g., an electrode, ultrasound transducer, or a sensor) to a therapy delivery device, e.g., a source of energy. The shape memory material is configured to facilitate the self-expansion of the portion (e.g., a distal portion) of the catheter from the delivery configuration to the deployed configuration. The catheter further includes an electrically insulative material that electrically insulates at least one filar from another filar.

In some examples, the catheters described herein may be useful for neuromodulation within a blood vessel or a body lumen other than a vessel, for extravascular neuromodulation, for non-renal-nerve neuromodulation, and/or for use in therapies other than neuromodulation.

In some examples, the disclosure describes a catheter comprising: an elongated body comprising a plurality of filars extending axially along the catheter, wherein each filar of the plurality of filars comprises: an outer layer formed from a first material; and an inner core radially inward of the outer layer and formed from a second material that is different from the first material, wherein one of the first material or the second material comprises an electrically conductive material and the other of the first material or the second material comprises a shape memory material; an electrically insulative material configured to electrically insulate at least one filar of the plurality of filars from another filar of the plurality of filars; and one or more electrodes electrically connected to the electrically conductive material of at least one filar of the plurality of filars.

In addition, in some examples, the disclosure describes a catheter comprising: an elongated body comprising a plurality of filars defining a tubular body, wherein each filar of the plurality of filars comprises: a first material; and a second material radially inward of the outer layer and different from the first material, wherein one of the first material or the second material comprises an electrically conductive material and the other of the first material or the second material comprises a shape memory material; an electrically insulative material configured to electrically insulate at least one filar of the plurality of filars from another filar of the plurality of filars, wherein the electrically insulative materials defines one or more openings; and one or more electrodes electrically connected to the electrically conductive material of at least one filar of the plurality of filars through the one or more openings of the electrically insulative material.

In some examples, the disclosure describes a method comprising: forming one or more openings in an electrically insulative material of an elongated body of a catheter, wherein the electrically insulative material is configured to insulate at least one filar of a plurality of filars of the elongated body from another filar of the plurality of filars, and wherein each filar of the plurality of filars comprises: an outer layer formed from a first material; and an inner core radially inward of the outer layer and formed from a second material that is different from the first material, wherein one of the first material or the second material comprises an electrically conductive material and the other of the first material or the second material comprises a shape memory material; and electrically connecting an electrode to the electrically conductive material of at least one filar of the plurality of filars through the one or more openings.

Further disclosed herein is a catheter that includes at least one therapeutic element and a plurality of electrically conductive filars configured to both electrically connect the at least one therapeutic element to a medical device, and to enable a portion of the catheter to expand radially outwards from a relatively low profile delivery configuration to a deployed configuration, wherein each filar of the plurality of filars includes an outer layer formed from a first material and an inner layer (also referred to herein as an inner core) radially inward of the outer layer and formed from a second material that is different from the first material, wherein one of the first material or the second material comprises an electrically conductive material and the other of the first material or the second material comprises a shape memory material.

The details of one or more examples are set forth in the accompanying drawings and the description below. Other features, objects, and advantages will be apparent from the description and drawings, and from the claims.

### BRIEF DESCRIPTION OF THE DRAWINGS

Reference is made to the attached drawings, wherein elements have the same reference numeral designations represent similar elements throughout.

FIG. 1 is a partial schematic illustration of an example catheter that includes a plurality of conductive and shape memory filars.

FIG. 2 is a conceptual diagram illustrating an example distal portion of the catheter of FIG. 1 positioned within a renal vessel.

FIG. 3A is a conceptual diagram illustrating a cross-sectional view of the example distal portion of the catheter of FIG. 2, the cross-section being taken along line B-B in FIG. 1.

FIG. 3B is a conceptual diagram illustrating a cross-sectional view of another example distal portion of the catheter of FIG. 2, the cross-section being taken along line B-B in FIG. 1.

FIG. 4 is a conceptual diagram illustrating a cross-sectional view of another example distal portion of the catheter of FIG. 2, the cross-section being taken along line B-B in FIG. 1.

FIG. 5 is a conceptual diagram illustrating a cross-sectional view of another example distal portion of FIG. 2, the cross-section being taken along line B-B in FIG. 1.

FIG. 6 is a conceptual diagram illustrating a cross-section view of a distal portion of the catheter of FIG. 2, the cross-section being taken along longitudinal axis A of the catheter.

FIG. 7 is a flow diagram illustrating an example method of using a catheter includes a plurality of electrically conductive filars.

FIG. 8 is a flow diagram illustrating an example method of manufacturing a catheter including a plurality of shape memory and electrically conductive filars and one or more therapeutic elements.

FIG. 9 illustrates an example technique for accessing a renal artery and modulating renal nerves with the catheter of FIG. 1.

FIG. 10 is a conceptual illustration of an example sympathetic nervous system (SNS) illustrating how the brain communicates with the body via the SNS.

FIG. 11 is an enlarged anatomic view of nerves innervating a left kidney to form the renal plexus surrounding the left renal artery.

FIG. 12 is an anatomic view of a human body depicting neural efferent and afferent communication between the brain and kidneys.

FIG. 13 is a conceptual view of a human body depicting neural efferent and afferent communication between the brain and kidneys.

FIG. 14 is an anatomic view of the arterial vasculature of a human.

FIG. 15 is an anatomic view of the venous vasculature of a human.

### DETAILED DESCRIPTION

The present disclosure describes catheters including at least one therapeutic element and a plurality of electrically conductive filars configured to both electrically connect the at least one therapeutic element to a medical device and to enable a portion of the catheter to expand radially outwards from a delivery configuration to a deployed configuration. The delivery configuration can be, for example, a relatively low profile configuration (e.g., a linear configuration), in which the portion of the catheter defines a smaller radial extent compared to when the portion of the catheter is in the radially expanded deployed configuration. The delivery configuration facilitates delivery of the at least one therapeutic element to a target tissue site in a in a hollow anatomical structure (e.g., a blood vessel) of a patient, while the deployed configuration facilitates engagement with and positioning of the at least one therapeutic element at the target tissue site. For example, the at least one therapeutic element can include a plurality of electrodes and, when the portion of the catheter is in the deployed configuration in a blood vessel, the electrodes may be spaced apart from each other circumferentially and/or longitudinally along the blood vessel wall. When the catheter is in the radially expanded configuration, at least part of the catheter body can define any suitable shape, such as, for example, a helical (or spiral) configuration, a loop, a radially expanded curved configuration that is not helical or a loop, or the like.

The plurality of filars of the catheter include both an electrically conductive material configured to electrically connect the at least one therapeutic element to a therapy delivery device and a shape memory material configured to facilitate the self-expansion of the catheter from the delivery configuration to the deployed configuration. Each filar includes an outer layer formed from a first material and an inner layer (also referred to herein as an inner core) radially inward of the outer layer and formed from a second material that is different from the first material. One of the first material or the second material comprises an electrically conductive material and the other of the first material or the second material comprises a shape memory material. For example, the inner core of the filar may comprise the shape memory material and the outer layer may comprise the electrically conductive material. As another example, the inner core may comprise the electrically conductive material and the outer layer may comprise the shape memory material. An electrically insulative material electrically insulates at least one filar from another filar.

The electrically conductive material has fewer shape memory properties than the shape memory material and, in some examples, is not a shape memory material. The shape memory material is less electrically conductive than the electrically conductive material and, in some examples, does not have sufficient electrically conductivity to act as a conduit through which an electrical signal can be delivered from a therapy delivery device to a therapeutic element of the catheter. In some examples, electrically conductive material is configured to resist deformation of the electrically conductive material in response to cyclic transformations of the shape memory material between the delivery configuration and the deployed configuration.

The plurality of filars extend axially along the catheter from a proximal portion of the catheter to a more distal portion of the catheter including the at least one therapeutic element. For example, the plurality of filars can extend linearly in a direction substantially parallel (e.g., parallel or within 5 degrees) to a longitudinal axis of the catheter and/or wrap around the longitudinal axis of the catheter to define a coil-like configuration. In some examples, the plurality of filars define part of an elongated catheter body of the catheter, e.g., are arranged to define a tubular shape defining one or more lumens, e.g., at least one of which is configured to receive a guide element, such as a guidewire.

In contrast to catheters that include shape memory elements that are separate from the electrical conductors of the catheter, the catheters described herein may have a reduced profile in both the delivery and deployed configurations (e.g., a smaller diameter in the case of a catheter having a circular cross-section) due to the incorporation of shape-memory materials and electrically conductive materials into a single elongated element (e.g., a filar). The examples described herein will primarily refer to a diameter of both a filar and a catheter, but the cross-sectional shape of the elements may be different in other examples in which case the examples will refer to the maximum cross-sectional dimensions of the cross-sectional shape.

Reducing the profile of a catheter in the delivery configuration may facilitate navigation of the catheter through certain blood vessels, e.g., to a target site in the vasculature from a radial artery. In addition, reducing the profile of a catheter in the deployed configuration may enable the deployed electrode length to be reduced, which can provide one or more advantages. For example, a smaller deployed electrode length may help target certain treatment sites in a patient, and/or avoid other tissue sites (e.g., helps prevent delivery of ablation energy to tissue site, such as a bifurcation in a blood vessel or another blood vessel location). As another example, a smaller deployed electrode length may enable the expandable portion of the catheter to assume certain shapes, e.g., a helix having a relatively small pitch or even a circular profile, which can facilitate delivery of ablation energy (e.g., to define a lesion) in a more circumferential pattern around a blood vessel wall in a direction orthogonal to a longitudinal axis of the blood vessel.

In some examples, incorporating both shape memory and electrically conductive material into a common filar may enable the expandable portion of the catheter including the plurality of filars to be less stiff. Reducing a stiffness of the expandable portion of the catheter may, for example, reduce the amount of force needed to transition the expandable portion from the deployed configuration to the delivery configuration to enable repositioning of the catheter within the patient or removal of the catheter from the patient. The force needed to transition the expandable catheter portion to the delivery configuration can be applied, e.g., via an outer sheath in which the expandable catheter portion is configured to be received, via a guidewire or other guide element configured to be received in a lumen of the catheter, or the any combination thereof or other techniques.

Incorporating shape memory and electrically conductive properties of an elongated element into a single filar, rather than using separate filars for the respective properties, can also help simplify a method of manufacturing the catheter, e.g., due to the reduce number of components and materials.

Although neuromodulation is primarily referred to herein, the catheters described herein may be used for medical procedures other than neuromodulation, including electrical stimulation therapy.

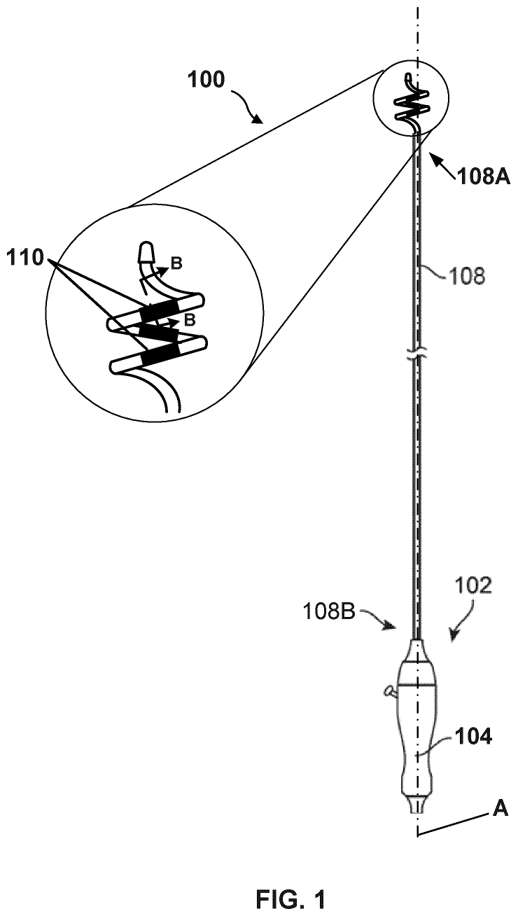

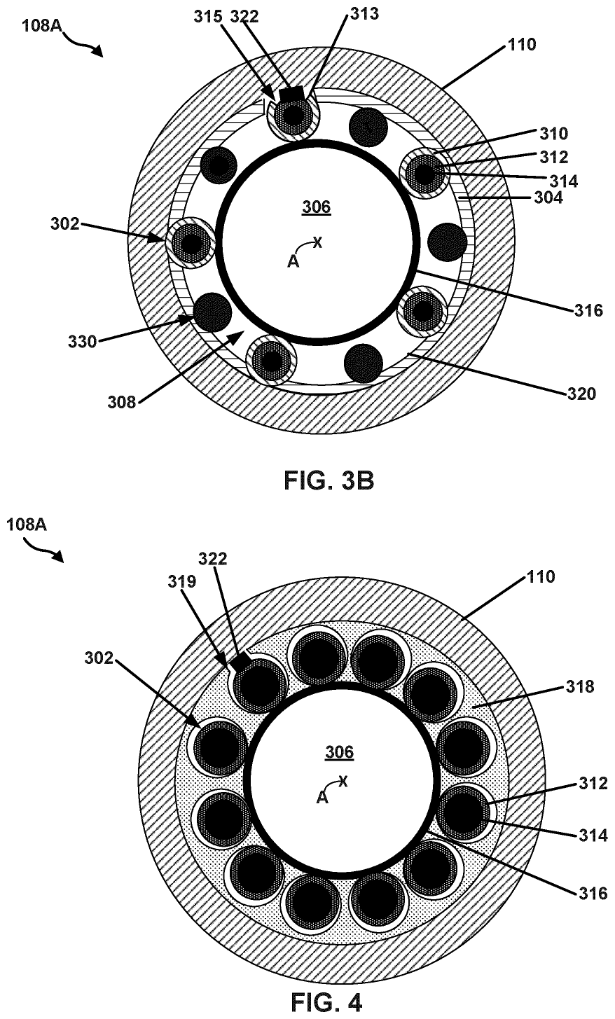

FIG. 1 is a schematic illustration of an example catheter 102 that includes a plurality of electrically conductive filars (shown in FIG. 3A) configured to both electrically connect at least one therapeutic element of catheter 102 to a medical device. Catheter 102 includes a handle 106, an elongated body 108 attached to handle 106, and at least one therapeutic element 110 carried by elongated body 108. Elongated body 108 includes a distal portion 108A and a proximal portion 108B. In the example of FIG. 1, all of the one or more therapeutic elements 110 are positioned at distal portion 108A of elongated body 108. In other examples, some or all of the therapeutic elements 110 can be positioned on a more proximal portion of catheter 102.

As used herein, the terms “distal” and “proximal” define a position or direction with respect to the treating clinician or clinician's control device (e.g., a handle assembly). “Distal” or “distally” can refer to a position distant from or in a direction away from the clinician or clinician's control device. “Proximal” and “proximally” can refer to a position near or in a direction towards the clinician or clinician's control device. For example, distal portion 108A of elongated body 108 refers to a portion of elongated body 108 at a position distant from the clinician and proximal portion 108B or elongated body 108 refers to a portion of elongated body 108 at a position near the clinician. In some examples, distal portion 108A is a distalmost portion of catheter 102 including a distal end of catheter 102.

Elongated body 108 of catheter 102 may have any suitable outer diameter, and the diameter can be constant along the length of elongated body 108 or may vary along the length of elongated body 108. In some examples, elongated body 108 can be 2, 3, 4, 5, 6, or 7 French or another suitable size.

The at least one therapeutic element 110 includes one or more elements configured to deliver energy (e.g., radiofrequency energy, ultrasound energy, electrical stimulation energy, or the like) from a medical device (also referred to herein as a therapy delivery device) to a target tissue site within a patient when distal portion 108A is positioned proximate the target tissue site. In some examples, the at least one therapeutic element 110 includes one or more electrodes distributed along the length of distal portion 108A of elongated body 108. For instance, the at least one therapeutic element 110 may include two, three, or four electrodes spaced from each other along longitudinal axis A of elongated body 108. Although a plurality of therapeutic elements 110 are referred to herein, in other examples, catheter 102 can include one therapeutic element, such as one electrode configured to be used in a monopolar configuration with a return electrode that is physically separate from catheter 102.

As discussed in further detail with reference to FIG. 3A, elongated body 108 includes a plurality of filars 302 configured to electrically connect therapeutic elements 110 to a medical device and configured to facilitate expansion of at least distal portion 108A from a relatively low profile delivery configuration to a radially expanded deployed configuration (shown in FIG. 1). The plurality of filars 302 may include two, three, or four or more filars 302. In other examples, elongated body 108 has only one filar 302.

Each filar 302 includes an outer layer formed from a first material and an inner layer formed from a second material different from the first material. The first and second materials have different chemical compositions and different properties. One of the first material or the second material includes a shape memory material and the other of the first material or the second material includes an electrically conductive material. In some of these examples, the shape memory material is not electrically conductive or is less electrically conductive than the electrically conductive material, and the electrically conductive material is not a shape memory material or exhibits less shape memory effect than the shape memory material of the filar. Catheter 102 also includes an electrically insulative material configured to electrically insulate the filars 302 from each other. For example, the electrically insulative material can fill a space between adjacent filars. In addition, or instead, in some examples, each filar has a respective layer of electrically insulative material radially outward of the outer layer.

The shape memory material of each filar is configured to transform at least a portion of elongated body 108 from a relatively smaller radial delivery configuration to a relatively larger radial extent deployed configuration, as the filar assumes a shape set configuration. Elongated body 108 can be held in a delivery configuration using any suitable mechanism, e.g., via maintaining the filars at a particular temperature range, via a guide member (e.g., a guidewire or an inner catheter) position in a lumen defined by elongated body 108 (e.g., a lumen defined by a tubular body defined by the plurality of filars 302) and configured to hold elongated body 108 in a linear or otherwise relatively low profile configuration, or via an outer sheath in which elongated body 108 is positioned. When the plurality of filars are permitted to recover a shape set configuration, (e.g., via heat activation, the retraction of the guide member from the inner lumen of elongated body 108 and/or via deploying elongated body 108 from an outer sheath), the plurality of filars cause at least a portion of catheter 102 (e.g., distal portion 108A in the example of FIG. 1) to transform from the delivery configuration to the deployed configuration. In the radially expanded deployed configuration, distal portion 108A is configured to position therapeutic elements 110 proximate a target tissue site, e.g., in apposition to the vessel wall. In some examples, in the deployed configuration, distal portion 108A defines a helical configuration, as shown in FIG. 1. In other examples, in the deployed configuration, distal portion 108A defines a curvilinear configuration that is not helical.

The electrically conductive material of the plurality of filars 302 is configured to electrically couple therapeutic elements 110 to a medical device. For example, the electrically insulative material of catheter 102 may define one or more openings to expose the electrically conductive material of the filar 302 and to enable the electrically conductive material to directly or indirectly (e.g., an electrically conductive interface material) contact a therapeutic element 110. In some examples, catheter 102 includes the same number of filars 302 and therapeutic elements 110, such that each filar 302 electrically connects to a respective therapeutic element 110 and enables separate electrical connection of each therapeutic element 110 to a medical device and independent control of each therapeutic element 110. In other examples, two or more therapeutic elements 110 are electrically coupled to the same filar 302 and/or two or more filars 302 are electrically connected to the same therapeutic element 110.

The plurality of filars 302 have any suitable arrangement. In some examples, the plurality of filars define a tubular body of elongated body 108, and the tubular body defines an inner lumen configured to receive a guide member (e.g., a guidewire or another catheter).

Distal portion 108A of elongated body 108 is configured to be advanced within a hollow anatomical structure (e.g., a blood vessel) of a human patient to locate therapeutic elements 110 at a target region (e.g., a target treatment site) within or otherwise proximate to the hollow anatomical structure. For example, elongated body 108 may be configured to position therapeutic element 110 within a blood vessel, a ureter, a urethra, a duct, an airway, or another naturally occurring lumen within the human body. The examples described herein focus on the anatomical structure being a blood vessel, such as a renal vessel, but it will be understood that similar techniques may be used with other hollow anatomical structures.

In certain examples, intravascular delivery of therapeutic elements 110 includes percutaneously inserting a guidewire (not shown in FIG. 1) into a vessel of a patient and moving elongated body 108 and/or therapeutic element 110 along the guidewire until therapeutic elements 110 reach a target treatment site (e.g., a renal artery). For example, distal portion 108A of elongated body 108 may define a passageway for engaging the guidewire for delivery of therapeutic element 110 using over-the-wire (OTW) or rapid exchange (RX) techniques. In other examples, catheter 102 can be a steerable or non-steerable device configured for use without a guidewire. In still other examples, catheter 102 can be configured for delivery via an inner catheter or sheath (not shown in FIG. 1), or other guide member. In some examples, catheter 102 can be configured to accept an inner catheter with an outer diameter of 6 Fr or less.

Once at the target treatment site, a medical device delivers therapeutic energy, such as RF energy or ultrasound energy, to the target treatment site, and/or senses a parameter, such as temperature or electrical activity, via the plurality of filars 302 and therapeutic elements 110. The energy can, for example, provide or facilitate neuromodulation therapy at the target treatment site. For ease of description, the following discussion will be primarily focused on delivering RF energy. It will be understood, however, that therapeutic element 110 may include elements or structures configured to deliver other types of therapy. For example, therapeutic element 110 may include ultrasound transducers configured to deliver ultrasound energy for ultrasound ablation of nerves near the vessel in which catheter 102 is positioned.

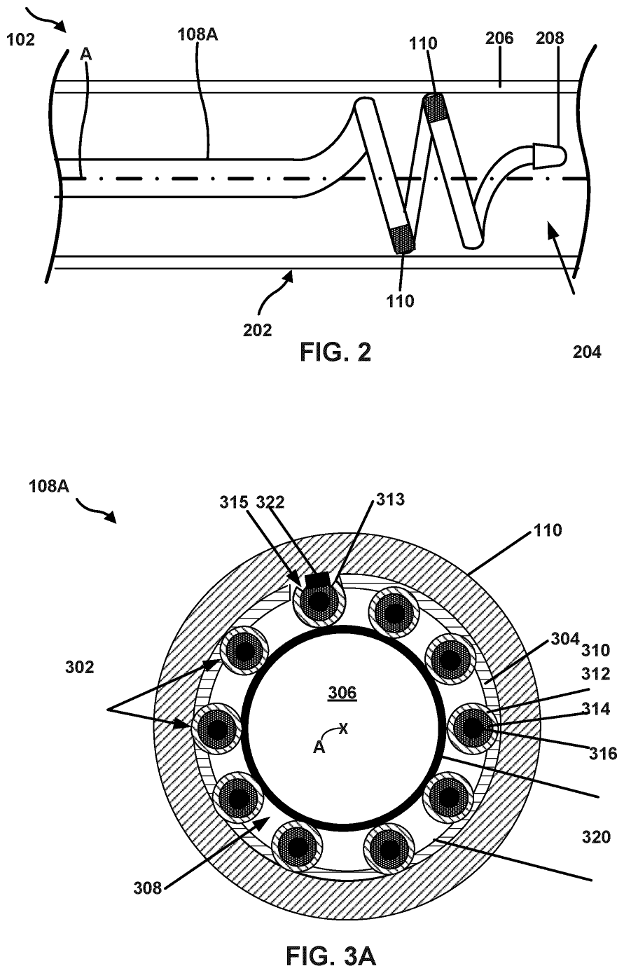

FIG. 2 is a conceptual diagram of distal portion 108A of elongated body 108 in an example deployed configuration within an example blood vessel 202. FIG. 2 also illustrates therapeutic elements 110 in apposition with vessel wall 206 of blood vessel 202. A medical device is configured to delivery energy (e.g., RF energy, ultrasound energy, microwave energy, electrical stimulation energy, or the like) to a target tissue site via filars 302 and therapeutic elements 110. In some examples in which blood vessel 202 is a renal vessel, the energy may be used to neuromodulate nerve tissue of the renal plexus adjacent to blood vessel 202 (e.g., by ablating the nerve tissue and creating lesions).

In addition to electrically connecting therapeutic elements 110 to a medical device, plurality of filars 302 are configured to enable distal portion 108A expand radially outwards in lumen 204 of vessel wall 206 to assume a radially expanded deployed configuration and position therapeutic elements 110 in apposition with vessel wall 206. In the example deployed configuration shown in FIG. 2, distal portion 108A defines a helical structure including a plurality of turns. The helix axis of the helical structure can be parallel to longitudinal axis A of catheter 102 (e.g., the helix axis may be coincident with longitudinal axis). For example, distal portion 108A may be configured such that, in the radially expanded helical configuration, the turns of the helix revolve around longitudinal axis A as elongated body 108 extends along the longitudinal axis A.

Distal portion 108A may trace any suitable number of helical revolutions in the deployed configuration. In the example shown in FIG. 2, distal portion 108A traces two complete helical revolutions around longitudinal axis A. In other examples distal portion 108A traces one helical revolution around longitudinal axis A, three or more helical revolutions around longitudinal axis A, or a non-integer number of helical revolutions. In yet other examples, distal portion 108A can assume another radially expanded shape that is not helical, e.g., an expanded loop, or the like.

In the example shown in FIG. 2, therapeutic elements 110 includes four electrodes (though only two are visible in the view shown in FIG. 2). In other examples, therapeutic elements 110 made include one electrode, two electrodes, three electrodes, or more than four electrodes. In some examples, filars 302 are configured to be separately electrically connect each therapeutic element 110 to an energy source, such that control circuitry of a therapy delivery device can independently control energy deliver to each therapeutic element 110. Each therapeutic element 110 may be electrically connected to the therapy delivery device through the conductive material of a different filar 302. In other examples, two or more therapeutic element 110 are electrically coupled together and are configured to be controlled by the control circuitry as a group. In such examples, one or more of the same filars may electrically connect the energy source to two or more therapeutic elements 110. In some examples, when distal portion 108A is in the deployed configuration, the electrical resistance of the electrically conductive material may be different from the electrical resistance of the same material when distal portion 108A is in the delivery configuration. However, the electrically conductive material can be selected such that the relative change in electrical resistance of the electrically conductive material between configurations is not large enough to alter the electrical conductivity of the filars 302.

Therapeutic elements 110 are positioned along longitudinal axis A and distal portion 108A. Therapeutic elements 110 may be positioned along distal portion 108A such that, when distal portion 108A is in the deployed configuration, therapeutic elements 110 are spaced around an inner perimeter (e.g., circumference) of lumen 204. In some examples where distal portion 108A includes two therapeutic elements 110, as illustrated in FIG. 2, the two therapeutic elements 110 may be positioned along distal portion 108A such that, when distal portion 108A is in the radially expanded helical configuration, each therapeutic element 110 is spaced approximately 90 degrees or 180 degrees apart from any other adjacent therapeutic element 110 around the inner perimeter of vessel wall 206.

In some examples, therapeutic elements 110 may also be positioned along distal portion 108A such that, when distal portion 108A is in the deployed configuration, the therapeutic elements 110 are located at a substantially similar longitudinal position within blood vessel 202. The longitudinal positioning of therapeutic elements 110 may be characterized using a deployed electrode length. As used herein, the deployed electrode length is a distance between, in the radially expanded helical configuration, a proximal-most point of a proximal-most therapeutic element 110 of the plurality of therapeutic elements 110 and a distal-most point of a distal-most therapeutic element 110 of the plurality of therapeutic elements 110. As discussed above, rather than having electrically conductive filars and shape memory filars that are separate from the electrically conductive filars, the plurality of filars 302 include both electrically conductive and shape memory properties. As a result, the overall maximum cross-sectional dimension of catheter 102 may be reduced. In this way, plurality of filars 302 can be configured to reduce the deployed electrode length of distal portion 108A, e.g., by reducing the outer diameter of elongated body 108. By reducing the outer diameter of elongated body 108, the plurality of filars 302 may allow distal portion 108A of elongated body 108 to complete more helical revolutions around longitudinal axis A for a given longitudinal distance (measured along longitudinal axis A).

A smaller deployed electrode length indicates a more longitudinally compressed deployment of therapeutic elements 110, and, thus, a more circular arrangement of therapeutic elements 110 when distal portion 108A of catheter 102 is in the radially expanded helical configuration. Conversely, a larger deployed electrode length indicates a less longitudinally compressed deployment of therapeutic elements 110, and, thus, a more elongated helical arrangement of therapeutic elements 110 when distal portion 108A is in the radially expanded helical configuration. In some examples, catheter 102 may define a deployed electrode length of between about 1 millimeters (mm) and about 10 mm, or about 1 mm and about 7 mm, or between about 1 mm and about 5 mm, or between about 1 mm and about 3 mm. The use of “about” or “approximately” in connection with a numerical value herein can refer to the exact value or within 5%-10% of the recited numerical value. Thus, for example, a deployed electrode length of about 10 mm can refer to a deployed electrode length of 10 mm or 10 mm +/−0.5 mm to 1 mm.

Catheter 102 may be configured to accommodate a variety of vessel diameters. For example, renal vessels may have a diameter between about 3 mm and about 8 mm. Other vessels may have other diameters. Because distal portion 108A of catheter 102 assumes a radially expanded helical configuration in a deployed state, distal portion 108A may accommodate different vessel diameters by assuming helices with different helical diameters. In this way, a single catheter 102 may be used to deliver therapy to vessels with different diameters, e.g., diameters in a range of between about 2 mm and about 10 mm.

In some examples, catheter 102 includes a distal tip 208 positioned at a distal end of distal portion 108A. Distal tip 208 may help control longitudinal extension and contraction of distal portion 108A when distal portion 108A transforms from the delivery configuration to the radially expanded deployed configuration, and vice versa. Distal tip 210 may be made of a biocompatible polymer. The polymer may include, for example, a thermoplastic, such as an elastomer. In some examples, the elastomer may include a polyurethane, a silicone, or a copolymer, such as a block copolymer including polyether block amide available under the trade name Pebax® available from Arkema S.A., Colombes, France. In some examples, distal tip 210 may be formed from a polymer with a relatively low Shore hardness so distal tip 210 presents a relatively atraumatic tip in case of contact between distal tip 210 and tissue.

In some examples, distal tip 210 may have a relatively extended longitudinal length. This may facilitate advancing of catheter 200 through vasculature of a patient, e.g., in the absence of a guidewire. In some examples, the length of distal tip 210, measured parallel to longitudinal axis 220, may be between about 5 mm and about 2 centimeters (cm).

In the example shown in FIG.2, catheter 102 is positioned within blood vessel 202. In some instances, catheter 102 may be positioned in a main renal artery, an accessory renal artery, or a branch vessel extending distally from a main renal artery or accessory renal artery. In other examples, catheter 102 may be positioned within another hollow anatomical structure (e.g., a different blood vessel, a non-blood vessel lumen).

FIG. 3A is a conceptual diagram illustrating a cross-sectional view of an example distal portion 108A of catheter 102 of FIG. 2, the cross-section being taken along line B-B in FIG. 1. FIG. 3A illustrates a plurality of filars 302 arranged around inner lumen 306 of catheter 102 and positioned radially inward of therapeutic element 110. The plurality of filars 302 define a tubular body 308, which at least partially defines inner lumen 306. Each filar 302 includes an outer layer 312 and an inner core 314 positioned radially inward of outer layer 312. In some examples, as illustrated in FIG. 3A, each filar 302 also includes a respective electrically insulative material 310 positioned radially outward of outer layer 312 and configured to electrically insulate the respective filar 302 from another filar 302 (e.g., an adjacent filar 302).

As illustrated in FIG. 3A, filars 302 are arranged in a circular shape around central longitudinal axis A of catheter 102 and around inner lumen 306. In other examples, depending on the configuration of catheter 102, the plurality of filars 302 may be arranged around inner lumen 306 of catheter 102 such that the plurality of filars 302 define a non-circular shape (e.g., an elliptical shape). Tubular body 308 at least partially defines inner lumen 306 configured to receive a guide member (e.g., a guidewire, an inner catheter, or the like) to facilitate the navigation of catheter 102 through vasculature of a patient or any other hollow anatomical structure. Catheter 102 of FIG. 3A includes twelve filars 302, but in other examples can include any other number filars 302, e.g., one filar, two, three, four or more filars 302. As illustrated in FIG. 3A, each filar 302 may have a tubular shape. In some examples, one of more filars 302 include an inner lumen positioned within filar 302.

In some examples, in the delivery configuration of elongated body 108, each filar 302 is parallel with longitudinal axis A as the plurality of filars 302 extends along the length of catheter 102. In other examples, in the delivery configuration, each filar 302 is coiled around longitudinal axis A, e.g., the plurality of filars 302 may advance radially (or circumferentially) around inner lumen 306 as the plurality of filars 302 extends longitudinally along longitudinal axis A. In some examples, such as in some examples in which each filar 302 includes a respective electrically insulative material 310, filars 302 are configured to move (e.g., longitudinally and/or laterally along elongated body 108) relative to another filar 302. That is, filars 302 may not be fixed (e.g., adhered, bonded, welded, or the like) to each other, but can be held together to define tubular body 308 using other structures, such as an outer catheter layer 304 and/or an inner liner 316. In other examples, however, relative positions of filars 302 are fixed, e.g., as described with reference to FIG. 4. In these examples, catheter 102 may or may not include outer catheter layer 304 and/or inner liner 316.

Each filar 302 is configured to combine shape memory properties and electrically conductive properties into a single component, rather than using separate components for each respective property. The plurality of filars 302 may help reduce the outer diameter of distal portion 108A of catheter 102 and the overall profile of catheter 102 by removing the need for the separate components used in other catheters and thereby removing the space occupied by the separate components. The separate components may include a separate outer insulative jacket (e.g., an elastane jacket) and separate shape memory wires (e.g., a plurality of bifilar thermocouple wires. The reduced profile of catheter 102 may facilitate the insertion of catheter 102 into a blood vessel or other hollow anatomical structure with a smaller inner diameter and reduce the deployed electrode length of distal portion 108A.

One of outer layer 312 or inner core 314 includes a shape memory material. The shape memory material can include, for example, a super-elastic material configured to transform catheter 102 from a substantially linear configuration to a radially expanded deployed configuration (e.g., a helical configuration). An example super-elastic material includes nickel titanium (e.g., nitinol or other nickel titanium alloy). The shape memory material may be shaped into a radially expanded configuration under the application of heat such that the shape memory material will retain a shape memory of the radially expanded configuration and self-transform to the radially expanded configuration. For example, catheter 102 may self-transform to the radially expanded deployed configuration when a clinician removes a guide member from inner lumen 306 of catheter 102 or removes a restraining force on the catheter, e.g., by deploying catheter 102 from an outer sheath. The guide member or other sheath prevents the transformation of filar 302 by restraining the radial movement of the plurality of filars 302. When the guide member is removed or the catheter is deployed from the outer sheath, the shape memory material of filar 302 is allowed to transform in response to the body heat of the patient. A clinician may then transform catheter 102 back to the substantially linear delivery configuration by restraining the shape memory material radially, e.g., by the re-insertion of a guide member into catheter 102 or reinsertion of catheter 102 into an inner lumen of the outer sheath. The shape memory material may be capable of substantial elastic deformation when an external force is exerted parallel to or orthogonal to longitudinal axis A of catheter 102.

The other of outer layer 312 or inner core 314 of each filar 302 includes an electrically conductive material. The electrically conductive material is configured to electrically connect one or more therapeutic elements 110 with an energy source proximal to distal portion 108A. The electrically conductive material is further configured to be mechanically connected to and elastically deform with the shape memory material of filar 302. For example, when the shape memory material (e.g., inner core 314) transforms to the radially expanded deployed configuration, the electrically conductive material (e.g., outer layer 312) elastically deforms to maintain contact with inner core 314 along the length of filar 302. The electrically conductive material includes an electrically conductive metal or metal alloy, such as, but not limited to, silver, copper, or platinum.

Catheter 102 also includes electrically insulative material 310 configured to electrically insulate each filar 302 from any other filar 302. In some examples, as illustrated in FIG. 3A, electrically insulative material 310 is a component of each filar 302 (e.g., a layer of the respective filar 302) and is positioned radially outward of outer layer 312. Electrically insulative material 310 may define an opening 313 configured to facilitate electrical connection between the electrically conductive material of the respective filar 302 and therapeutic element 110. Electrically insulative material 310 can be formed from any suitable electrically insulative material, such as, but not limited to, an electrically insulative thermoplastic material (e.g., parylene or a polymer heat shrink material).

Catheter 102 includes an outer catheter layer 304 (e.g., an outer jacket) and therapeutic element 110 (e.g., an electrode, an ultrasound transducer, or the like). Outer catheter layer 304 covers filars 302 and helps restrain movement of plurality of filars 302. In some examples, outer catheter layer 304 may be a tubular sleeve positioned radially outward of and around an outer perimeter of the plurality of filars 302. In addition, outer catheter layer 304 defines a relatively smooth outer surface of catheter 102. In some examples, together with filars 302, outer catheter layer 304 is configured to provide elongated body 108 with desirable stiffness characteristics, e.g., provide elongated body 108 with suitable column strength and rotational stiffness to facilitate navigation of elongated body 108 through vasculature of a patient. Outer catheter layer 304 may be positioned radially outward from the plurality of filars 302. In some examples, outer catheter layer 304 may be coincidental with and/or radially inward of therapeutic element 110. Outer catheter layer 304 may be formed from any suitable material or combination of materials, such as, but not limited to, one or more of nylon or a thermoplastic such as polyethylene terephthalate (PET), parylene, polyvinyl chloride (PVC), polyethylene, ethylene chlorotrifluoroethylene (ECTFE), or polyvinylidene fluoride (PVDF).

Outer catheter layer 304 defines one or more openings 315 configured to facilitate electrical connection between therapeutic elements 110 and the electrically conductive material of one or more of filars 302. Openings 315 are aligned with respective openings 313 defined by the electrically insulative material 310 of filars 302. If the electrically conductive material of filars 302 is inner core 314, then each opening 315 defined by outer catheter layer 304 and each opening 313 defined by the electrically insulative material 310 of filars 302 are also aligned with a respective opening defined through an outer layer 312 of a filar 302. The openings 313, 315 provide electrically conductive pathways from therapeutic elements 110 to the electrically conductive material of filars 302. An electrically conductive material 322 (e.g., a flowable electrically conductive material) may be placed within openings 313 and openings 315 to facilitate electrical connection between therapeutic element 110 and filar 302. Electrically conductive material 322 can be the same as or different from the electrically conductive material of filars 302. Electrically conductive material 322 may include, for example, one or more of solder (e.g., a silver solder), a conductive adhesive, or an electrically conductive mechanical attachment device (e.g., a mechanical crimp). In other examples, electrically conductive material 322 may be a portion of the electrically conductive material of one or more filars 302 and/or a portion of therapeutic element 110 that is crimped together or that is melted and reformed into an appropriate shape, e.g., via a welding process.

In some examples, catheter 102 also includes an inner liner 316 positioned radially inward of the plurality of filars 302. Inner liner 316 defines an inner lumen 306 and helps define a relatively smooth inner surface of catheter 102, and, in some cases, defines a lubricious inner surface to help facilitate passage of a medical device (e.g., a guidewire) through inner lumen 306. Inner liner 316 can be formed from any suitable material, such as a polymer, including a thermoplastic elastomer, such as, but is not limited to, a block copolymer including polyamide and polyether (e.g., Pebax®) or polytetrafluoroethylene (PTFE).

FIG. 3B is a conceptual diagram illustrating a cross-sectional view of another example distal portion 108A of the catheter 102 of FIG. 2, the cross-section being taken along line B-B in FIG. 1. In the example illustrated in FIG. 3B, catheter 102 includes a plurality of non-conductive filars 330 in addition to filars 302. Non-conductive filars 330 may include a shape memory material and, in some cases, an electrically insulative material, e.g., those shape memory and electrically insulative materials described with reference to filars 302. In some examples, non-conductive filars 330 may be configured to perform the same functions as filars 302 with the exception of conducting electricity. Filars 302 and non-conductive filars 330 are formed from different materials. For example, while both filars 302 and non-conductive filars 330 can include a shape memory material and, in some cases, an electrically insulative material, filars 330 do not include an electrically conductive material.

In some examples, as illustrated in FIG. 3B, each filar 302 may be separated from another filar 302 by a non-conductive filar 330. In other examples, different arrangements of filars 302 and non-conductive filars 330 around inner lumen 306 may be used in catheter 102. In some examples, non-conductive filar 330 do not include electrically insulative material 318 positioned radially outward of non-conductive filar 330.

FIG. 4 is a conceptual diagram illustrating a cross-sectional view of another example distal portion 108A of catheter 102 of FIG. 2, the cross-section being taken along line B-B in FIG. 1. In the example illustrated in FIG. 4, instead of or in addition to each filar 302 having a respective electrically insulative material (radially outwards of the respective outer and inner layers), catheter 102 includes an electrically insulative material 318 filling space between filars 302. Electrically insulative material 318 is radially outward of an inner liner 316 and radially inward of therapeutic element 110. In some examples, where outer layer 312 is the electrically conductive material, electrically insulative material 318 may fill the entire space between filars 302. Such a close contact between electrically insulative material 318 and filars 302 may help improve the overall profile of catheter 102.In other examples, there may be a gap between electrically insulative material 318 and at least some or all of the filars 302. The gap may help improve flexibility of catheter 102. In examples in which electrically insulative material 318 extends along the length of catheter 102, electrically insulative material 318 can help maintains the profile of catheter 102 along longitudinal axis A by constraining the relative movement of the plurality of filars 302. When distal portion 108A transforms to a radially extended deployed configuration, electrically insulative material 318 elastically deforms and remains in the space between filars 302.

Electrically insulative material 318 can comprise any suitable material, such as, but not limited to, a reflowed polymer, such as Pebax. The reflowed polymer is configured to transition from a solid when the temperature of the polymer exceeds its glass transition temperature, enabling the polymer to flow into space between adjacent filars 302 and fill the space.

In some examples, electrically insulative material 318 increases stiffness of catheter 102 along the longitudinal axis A and may aid the advancing and/or retracting of catheter 102 through vasculature (or other hollow anatomical structure) of a patient. For example, electrically insulative material 318 may provide the desired stiffness to provide catheter 102 with the pushability/pullability, improved torque response, and even reduce friction during navigation of catheter 102 through vasculature of a patient to a target treatment site.

One or more openings 319 are defined through electrically insulative material 310 to facilitate the electrical connection between therapeutic element 110 and the electrically conductive material of a filar 302. If the electrically conductive material of filars 302 is outer layer 312, then filars 302 may not define an opening that aligns with openings 319 in electrically insulative material 310, e.g., as shown in FIG. 4. As shown in FIG. 4, electrically conductive material 322 can be positioned in opening 319 (e.g., fill the opening 319) to define an electrically conductive pathway between therapeutic element 110 and outer layer 312 of a filar 302.

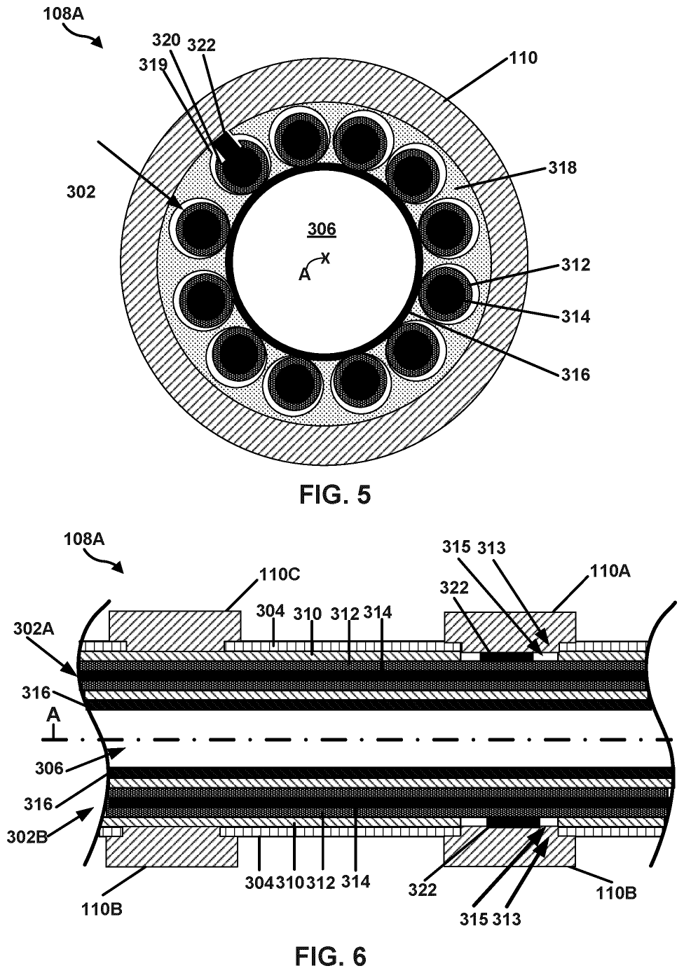

If the electrically conductive material of filars 302 is inner core 314, then filars 302 may define an opening 320 that aligns with a respective insulative material opening 319 in order to enable therapeutic element 110 to electrically connect to inner core 314, e.g., as shown in FIG. 5. FIG. 5 is a conceptual diagram illustrating a cross-sectional view of another example distal portion 108A of FIG. 2, the cross-section being taken along line B-B in FIG. 1. As illustrated, openings 320 may facilitate the electrical connection between therapeutic element 110 and an electrically conductive inner core 314 of a filar 302. As shown in FIG. 5, electrically conductive material 322 can fill the opening 313 to define an electrically conductive pathway between therapeutic element 110 and filar 302. In some examples, as illustrated in FIG. 5, catheter 102 includes electrically insulative material 118 filling space between filars 302 in the manner illustrated in FIG. 4. In other examples, each filar 302 may include a respective electrically insulative material 310, e.g., in the manner illustrated in FIG. 3A, in which case opening 320 may be defined by openings in the respective electrically insulative material 310 and outer layer 312 of each filar 302.

As shown in FIGS. 4 and 5, in examples in which an electrically insulative material 318 fills the space between filars 302, catheter 102 can include inner liner 316 radially inward of electrically insulative material 318. In some examples in which an electrically insulative material 318 fills the space between filars 302, catheter 102 does not include outer catheter layer 304 (FIG. 3A). The electrically insulative material 318 may sufficiently hold filars 302 in the desired shape (e.g., a tubular body), such that outer catheter layer 304 can be eliminated, thereby further reducing the cross-sectional dimension (e.g., diameter) of catheter 102. In other examples, catheter 102 includes an outer catheter layer 304 radially outward of electrically insulative fill material 318, in which case openings may be defined through outer catheter layer 304 to provide an electrically conductive pathway between therapeutic element 110 and at least one filar 302.

As discussed above, in some examples, each therapeutic element (e.g., therapeutic element 110) of catheter 102 may be electrically connected to a separate filar 302. In Some examples, therapeutic elements 110 may also be positioned at different longitudinal positions along distal portion 108A of catheter 102. FIG. 6 illustrates an example distal portion 108A including therapeutic elements 110 positioned at different longitudinal positions and electrically connected to separate filars 302.

FIG. 6 is a conceptual diagram illustrating a cross-section view of a longitudinal section of an example distal portion 108A of the catheter 102 of FIG. 2, the cross-section being taken along longitudinal axis A and through a longitudinal center of catheter 102. In the example illustrated in FIG. 6, each filar 302 includes a respective electrically insulative material 310 in the manner illustrated in FIG. 3A. In other examples, distal portion 108A may include electrically insulative material 318 filing around filars 302 as illustrated in FIGS. 4 and 5.

As illustrated in FIG. 6, electrically insulative material 310 and outer catheter layer 304 define one or more openings 313 configured to connect an electrically conductive material of filars 302 (e.g., outer layer 312) to therapeutic elements 110, e.g., via electrically conductive material 322. To facilitate depiction of openings 313, 315, electrically conductive material 322 is not shown in FIG. 6 as filling openings 313, 315. In some examples however, electrically conductive material 322 fills openings 313, 315.

In FIG. 6, plurality of filars 302 includes at least filars 302A, 302B and the at least one therapeutic element 110 includes at least therapeutic elements 110A, 110B. Filars 302A, 302B have different circumferential positions in the example of FIG. 6 and extend linearly along longitudinal axis A. In other examples, filars 302A, 302B can have another arrangement relative to longitudinal axis A, e.g., may coil around longitudinal axis A. Therapeutic elements 110A, 110B are physically separate from each other and displaced from each other along longitudinal axis A. In the example shown in FIG. 6, therapeutic elements 110A, 110B are electrically connected to different filars 302A, 302B, respectively, and are independently activatable. That is, in some examples, a first filar 302A is electrically connected to a first therapeutic element 110A, e.g., via electrically conductive material 322, and not to a second therapeutic element 110B, while a second filar 302B is electrically connected to second therapeutic element 110B and not to first therapeutic element 110A. First therapeutic element 110A is electrically disconnected from second therapeutic element 110B that such an electrical circuit formed by first filar 302A and first therapeutic element 110A is separate from an electrical circuit formed by second filar 302B and second therapeutic element 110B.

At least a portion of each therapeutic element (e.g., first therapeutic element 110A) may be partially electrically insulated from corresponding filar (e.g., first filar 302A), e.g., by outer catheter layer 304 and/or outer layer 312. In other examples, the entire length (measured along longitudinal axis A) of first therapeutic element 110A is electrically connected to filar 302A, e.g., to outer layer 312 or inner core 314.

An electrically conductive pathway is defined from therapeutic element 110 (e.g., a radially inward surface of therapeutic element 110) to the electrically conductive material of filar 302 (e.g., outer layer 312 or inner core 314). In the example shown in FIG. 6, the electrically conductive pathway is defined is defined through outer catheter layer 304 by an opening 313 and through an electrically insulative material 310 of a filar 302 by opening 315. In examples in which the electrically conductive material of filar 302 is inner core 314, opening 315 extends through outer layer 312 of the filar 302. The one or more openings 313, 315 may have any suitable shape, such as circular, rectangular, or of any other shape. In some examples, a portion of outer layer 312 is configured to enter into opening 313 to electrically connect with therapeutic element 110. In other examples, an electrically conductive material 322 is placed within openings 313 to facilitate the electrical connection between therapeutic element 110 and filar 302.

Filar 302B is electrically connected to therapeutic element 110B in a manner similar to that described with reference to therapeutic element 110A and filar 302A.

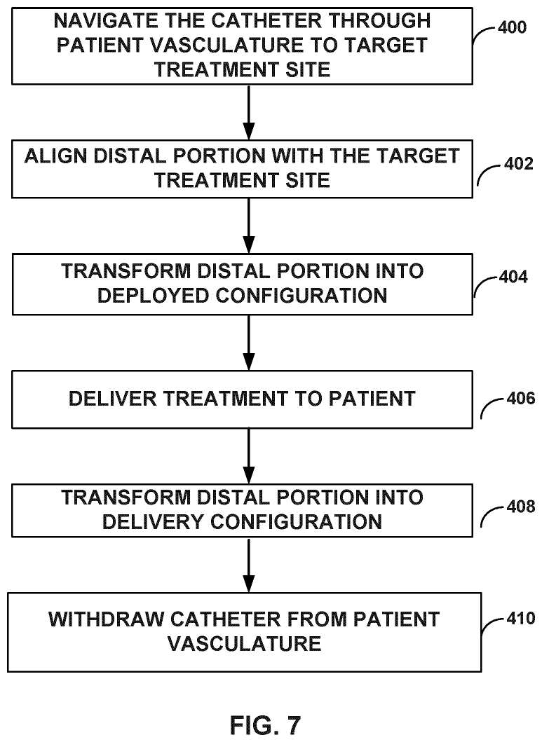

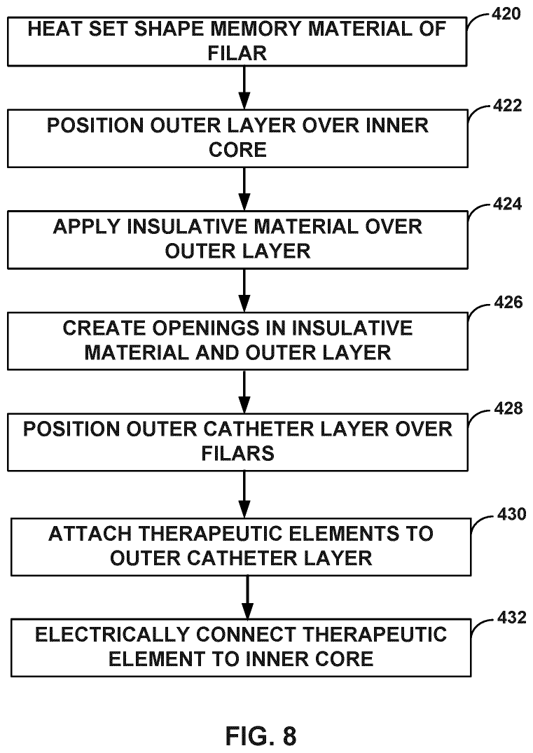

FIG. 7 is a flow diagram illustrating an example method of using a catheter that includes a plurality of electrically conductive filars. The techniques of FIGS. 7 and 8 will be described with reference to catheter 102 of FIG. 1. However, a person having ordinary skill in the art will understand that the technique of FIGS. 7 and 8 may be performed using any of the catheters described herein, and the catheters described herein may be used for other techniques.

A clinician navigates catheter 102 through vasculature of a patient to a target treatment site (400). In other examples, the clinician may navigate catheter 102 through another hollow anatomical structure of the patient to the target treatment site. The clinician may navigate catheter 102 to the target treatment site through an access site in a femoral artery, a brachial artery, a radial artery, or the like. The clinician may navigate catheter 102 through patient vasculature using handle 104 (FIG. 1) and one or more guiding members (e.g., a guidewire, an outer sheath, another catheter, or the like). The target treatment site may be any location at which the clinician intends to deliver energy (e.g., RF energy, ultrasound energy, microwave energy, electrical stimulation energy, or the like) such as, but is not limited to, vessel wall 206 (FIG. 2). In some examples, the clinician may select a plurality of target treatment sites for treatment. When the clinician navigates catheter 102 to the target treatment site, catheter 102 is configured in a relatively low profile delivery configuration, such as a linear configuration.

The clinician aligns distal portion 108A of elongated body 108 with the target treatment site (402). The clinician may align distal portion 108A with the target treatment site such that when distal portion 108A transforms into a radially expanded deployed configuration, one or more therapeutic elements 110 contact vessel wall 206 at the target treatment site. In some examples, the clinician aligns therapeutic element 110 with the target treatment site after transforming distal portion 108A into a partially deployed or completely deployed configuration, e.g., by advancing catheter 102 along longitudinal axis A and/or rotating catheter 102 around longitudinal axis A.

The clinician transforms distal portion 108A from the delivery configuration into the radially expanded deployed configuration (e.g., the helical configuration as shown in FIGS. 1, 2) (404). The clinician transforms distal portion 108A by retracting a guide member (e.g., a guidewire, an inner catheter, or the like), retracting an outer sheath and/or distally advancing catheter 102 out of the outer sheath, or through another similar technique.

Once distal portion 108A is transformed into the deployed configuration, the clinician controls a therapy delivery device to delivery energy to the target treatment site via therapeutic elements 110 (406). In some examples, the energy is configured to ablate tissue or otherwise modulate activity of nerves proximate the target treatment site. In other examples, instead of or in addition to delivery energy to the target treatment site, the clinician controls a sensing device to sense a parameter (e.g., temperature, pressure, electrical activity, or the like) via one or more therapeutic elements 110. After energy is delivered to the target treatment site and/or the parameter sensing is complete, the clinician transforms distal portion 108A back to the delivery configuration (408), e.g., by performing the reverse operation used to transform distal portion 108A to the deployed configuration. The clinician then withdraws catheter 102 from patient vasculature (410). In some examples, the clinician may, instead of withdrawing catheter 102 from patient, navigate catheter 102 to a second target treatment site and repeat example method of FIG. 7.

FIG. 8 is a flow diagram illustrating an example method of forming a catheter including a plurality of filars, each filar including both shape memory and electrically materials. The technique of FIG. 8 will be primarily described with reference to a filar having an inner core having a shape memory material and not an electrically conductive material, and an outer layer having an electrically conductive material and not a shape memory material. In other examples, the technique of FIG. 8 may be used to form other catheters including a plurality of filars, and may also be used to form a catheter including a filar including an inner core including an electrically conductive material and not a shape memory material, and an outer layer including a shape memory material and not an electrically conductive material.

In the technique of FIG. 8, a manufacturer shapes a shape memory material of plurality of filars 302 under heat into a desired radially expanded configuration for distal portion 108A of catheter 102 (420). In the example shown in FIG. 8, inner core 314 of filars 302 includes the shape memory material. The shape memory material is shaped under heat such that when heat is re-applied to the shape memory material, the material will self-transform into the radially expanded configuration. Inner core 314 may be shaped by heating inner core 314 to a set temperature (e.g., from about 400° C. to about 500° C. for nitinol) for a set period (e.g., less than 10 minutes), shaping the heated inner core 314 into the radially expanded configuration, and rapidly cooling inner core 314. By heating and rapidly cooling inner core 314, inner core 314 will normally assume a first material structure (e.g., a martensitic structure) but will assume a second material structure (e.g., an austenite structure) with the application of heat and transform into the radially expanded configuration. The radially expanded configuration may be a helical configuration, a loop configuration, or the like.

The manufacturer positions outer layer 312 over inner core 314 (422). In examples in which outer layer 312 is formed from an electrically conductive material and inner core 314 is formed from a shape memory material, outer layer 312 is substantially thinner than inner core 314 to reduce the stiffness of filar 302. In addition, the relative thin outer layer 312 may help reduce the effect of outer layer 312 on the shape memory properties of inner core 314, e.g., to reduce the resistance of filar 302 to transform, under shape memory of inner core 314, into the radially expanded deployed configuration. A thickness of outer layer 312 and inner core 314 can be measured in a direction orthogonal to longitudinal axis A. In some examples, to help achieve the relative thin outer layer 312, the electrically conductive material may be applied to an outer surface of inner core 314 using a three-dimensional (3D) printing or additive manufacturing technique, e.g., by using a free flowing electrically conductive material (e.g., a silver ink) as a filament.

In examples in which outer layer 312 of a filar 302 is formed from a shape memory material and inner core 314 of the filar 302 is formed from the electrically conductive material, the shape memory material may be shape set after application of the shape memory material over the electrically conductive inner core 314. The shape memory material can be applied to the electrically conductive inner core 314 using any suitable technique, including those described above with respect to the shape memory outer layer 312.

In accordance with the technique shown in FIG. 8, the manufacturer applies an electrically insulative material 310 (FIG. 3A) or 318 (FIG. 4) over outer layer 312 (424). Electrically insulative material 310, 318 may be applied over the entire outer layer 312 of filar 302 or to a portion of outer layer 312 (e.g., a portion of outer layer 312 corresponding to distal portion 108A of catheter 102). A melting temperature of electrically insulative material 310, 318 may be lower than a temperature used to heat set the shape memory material of filars 302 into a desired shape (e.g., a helical shape). Thus, in some examples, electrically insulative material 310, 318 is applied over outer layer 312 after outer layer 312 and inner core 314 have cooled to below the melting temperature of electrically insulative material 310, 318 and/or other threshold temperature.

Electrically insulative material 310 can be applied to outer layer 312 of filar 302 using any suitable technique. In some examples, each filar 302 includes a respective electrically insulative material layer 310, e.g., as shown in FIG. 3A. That is, each filar 302 includes a respective electrically insulative material layer 310. In some of these examples, particulates of electrically insulative material 310 are condensed and formed into a solid coating over outer layer 312 of a filar 302, e.g., through a vapor deposition technique. In other examples, electrically insulative material 310, in a liquid form, is applied over outer layer 312 and cooled into a solid, e.g., through a polymer reflow technique, dip coating technique, a vapor deposition technique (e.g., parylene vapor deposition), via a heat shrink tubing formed from an electrically insulative material, or the like. In other examples, preformed electrically insulative material 310 (e.g., a tubular body made of electrically insulative material 310) is positioned around outer layer 312 and then bonded to outer layer 312, e.g., through the application of heat in a heat shrink technique.

In some examples, in addition to or instead of each filar 302 including a respective electrically insulative material layer 310, an electrically insulative material 318 is positioned in spaces between filars 302 and around filars 302, e.g., as shown in FIG. 4. In these examples, electrically insulative material 318 is reflowed around filars 302. For example, after filars 302 are arranged to define a tubular body 308 or the like, an electrically insulative material 318 (e.g., in the form of a tube or the like) can be positioned around filars 302 and heated, such that the electrically insulative material 318 melts and flows into spaces between filars 302. The resulting structure may define a tubular body in some examples.

After electrically insulative material 310, 318 is applied over outer layer 312, the manufacturer removes portions of electrically insulative material 310 to define a pathway through which at least one therapeutic element 110 can electrically connect to the electrically conductive material of filar 302 (426). For example, the manufacturer may remove electrically insulative material 310, 318 to define opening 313. In some examples, a single opening 313 is formed on each filar 302. In other examples, the manufacturer may form multiple (e.g., two or more) openings 313 on each filar 302. Openings 313 can be formed using any suitable technique, such as via etching or cutting. In some examples in which each filar 302 has a respective electrically insulative layer 310, the manufacturer can form opening 313 by at least applying a mask (e.g., a heat shrink mask) over a portion of outer layer 312 prior to applying electrically insulative material 310 and removing the mask once electrically insulative material 310 is applied over the rest of outer layer 312. In other examples, the manufacturer may remove a portion of electrically insulative material 310, 318 through one or more instruments, e.g., through laser etching using a laser etching device, a mechanical etching device, such as a bladed instrument, or the like.

As discussed above, catheter 102 can include an outer catheter layer 304 in some examples. Thus, in some examples of the method of FIG. 8, the manufacturer positions outer catheter layer 304 radially outward of the plurality of filars 302 (428). Outer catheter layer 304 may be formed of a thermoplastic, such as, but not limited to PET, and may define a relatively smooth outer surface. Outer catheter layer 304 can be positioned over the plurality of filars 302 using any suitable technique, such as heat shrinking, dip coating, polymer reflow, chemical vapor deposition or the like. In some examples, outer catheter layer 304 is formed from a single unitary body, while in other examples, outer catheter layer 304 includes multiple portions, such as multiple longitudinally-extending portions (e.g., tubes).

In some examples, the manufacturer may create openings (e.g., openings 315) in outer catheter layer 304 prior to positioning outer catheter layer 304 over the plurality of filars 302 and then align openings 313 in electrically insulative material 310, 318 of a filar 302 with a corresponding opening 315. In other examples, the manufacturer may define openings 315 in outer catheter layer 304 and openings 313 in electrically insulative material 310 of filars 302 during the same manufacturing step, such as by cutting, etching, or the like through both outer catheter layer 304 and electrically insulative material 310, 318 at the same time such that each outer catheter layer opening 315 is aligned with a respective insulative material opening 313.

In some examples, catheter 102 includes inner liner 316 positioned radially inward of the plurality of filars 302. Thus, in some examples of the method of FIG. 8, the manufacturer may position filars 302 over inner liner 316. For example, the manufacturer may position inner liner 316 over a mandrel, position filars 302 over inner liner 316, and then position outer catheter layer 304 over filars 302. In some examples, to position filars 302 over inner liner 316, filars 302 can be arranged linearly along longitudinal axis A, can be arranged to wrap around longitudinal axis A while extending longitudinally (e.g., coiled about longitudinal axis A), or have any other suitable arrangement. When extending linearly along longitudinal axis A, filars 302 may be substantially parallel to each other (e.g., parallel or nearly parallel to the extent permitted by manufacturing tolerances).

In examples in which catheter 102 does not include inner liner 316, the plurality of filars 302 may define a tubular body 308. For example, if electrically insulative material 318 is reflowed around filars 302, e.g., as shown in FIG. 4, then an inner surface of the tubular body 308 defined by the filars 302 and electrically insulative material 318 may define inner lumen 306.

The manufacturer also attaches one or more therapeutic elements 110 to outer catheter layer 304 (430). Each therapeutic element 110 is attached to outer surface of outer catheter layer 304, e.g., with an adhesive, attachment device or the like, and is aligned with opening 313 and opening 315 such that the electrically conductive material of a filar 302 electrically connects with the respective therapeutic element 110 through opening 313 and opening 315, e.g., through an electrically conductive material placed within opening 313 and opening 315. In this way, the manufacturer may electrically connect each therapeutic element 110 to a filar 302 (432), e.g., through an electrically conductive material within opening 313, through a portion of outer layer 312 that extends into opening 313, or the like.

In examples in which inner core 314 of filars 302 are formed from the electrically conductive material and outer layer 312 is formed from the shape memory material, the method of FIG. 8 may also include forming an opening 313 through outer layer 312 to define a pathway to electrically connect at least one therapeutic element 110 to a respective inner core 314. The opening 313 through outer layer 312 is aligned with a respective opening 315 defined in electrically insulative layer 310.

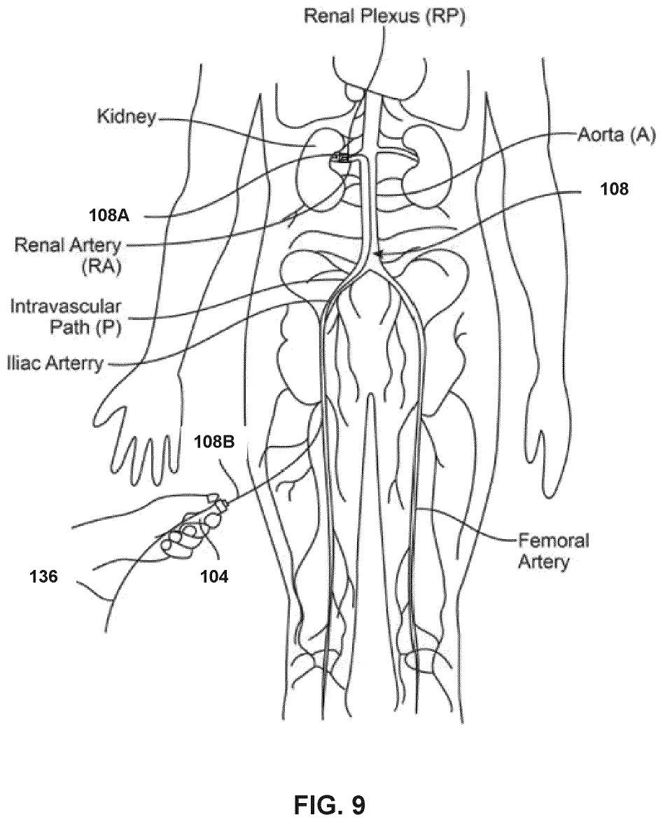

As discussed above, in some examples, catheter 102 may be used to access and modulate renal nerves through renal denervation. FIGS. 9-15 illustrates human anatomy relevant to renal denervation and example techniques for modulating renal nerves with catheter 102. In other examples, catheter 102 may be used with other medical procedures.

FIG. 9 illustrates an example technique for accessing a renal artery and modulating renal nerves with the catheter 102 of FIG. 1. While FIG. 9 illustrates the use of catheter 102 for renal neuromodulation, catheter 102 may be used for other therapies and treatments within another blood vessel or other hollow anatomical body within the human body. Catheter 102 is configured to delivery energy (e.g., radiofrequency energy, ultrasound energy, electrical stimulation energy, or the like) to one or more target treatment sites within a renal vessel. Catheter 102 provides access to the renal plexus (RP) through an intravascular path (P), such as a percutaneous access site in the femoral (illustrated), brachial, radial, or axillary artery to the target treatment sites within a respective renal artery (RA). By manipulating proximal portion 108B of elongated body 108 from outside the intravascular path (P), a clinician may advance at least distal portion 108A of elongated body 108 through the sometimes-tortuous intravascular path (P) and remotely manipulate distal portion 108A (FIG. 1) of elongated body 108. Distal portion 108A may be remotely manipulated by the clinician using the handle 104.

In the example illustrated in FIG. 9, distal portion 108A is delivered intravascularly to the treatment site using a guide member 136 in an over-the-wire (OTW) technique. Guide member 136 may be internal to catheter 102 (e.g., a guide wire, inner catheter, or the like) or external to catheter 102 (e.g., an outer sheath or the like). Catheter 102 may define a passageway for receiving guide member 136 for delivery of catheter 102 using either an OTW or a rapid-exchange (RX) technique. At the treatment site, guide member 136 can be at least partially withdrawn or removed relative to catheter 102 and distal portion 108A can transform into a radially expanded configuration (e.g., a helical configuration, a spiral configuration, or the like) for delivering energy. The shape memory material of filars 302 within distal portion 108A is configured to self-expand to the radially extended configuration upon being released from guide member 136 in response to the body heat of the patient. In other examples, elongated body 108 may be self-steerable such that therapeutic elements 110 may be delivered to the target treatment site without the aid of guide member 136.

Renal modulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for a period of months, years, or decades) or short-term (e.g., for periods of minutes, hours, days, or weeks). Renal neuromodulation is expected to contribute to the systemic reduction of sympathetic tone or drive and/or benefit at least some specific organs and/or other bodily structures innervated by sympathetic nerves. Accordingly, renal neuromodulation is expected to be useful in treating clinical conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end state renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced or induced in another suitable manner through the delivery of energy (RF energy, ultrasound energy, microwave energy, or the like). The target treatment site can be within or otherwise proximate to a renal lumen (e.g., a renal artery, a ureter, a renal pelvis, a major renal calyx, a minor renal calyx, or another suitable structure), and the target treatment site can include tissue at least proximate to a wall of the renal lumen. For example, with regard to a renal artery, a treatment procedure can include modulating nerves in the renal plexus, which lay intimately within or adjacent to the adventitia of the renal artery. The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of the target treatment devices and associate methods for achieving renal neuromodulation via intravascular access and impose specific design requirements for such devices. Specific design requirements may include accessing the renal artery, positioning distal portion 108A within the renal artery, delivering the therapy to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operated through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic neurons).

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up-and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

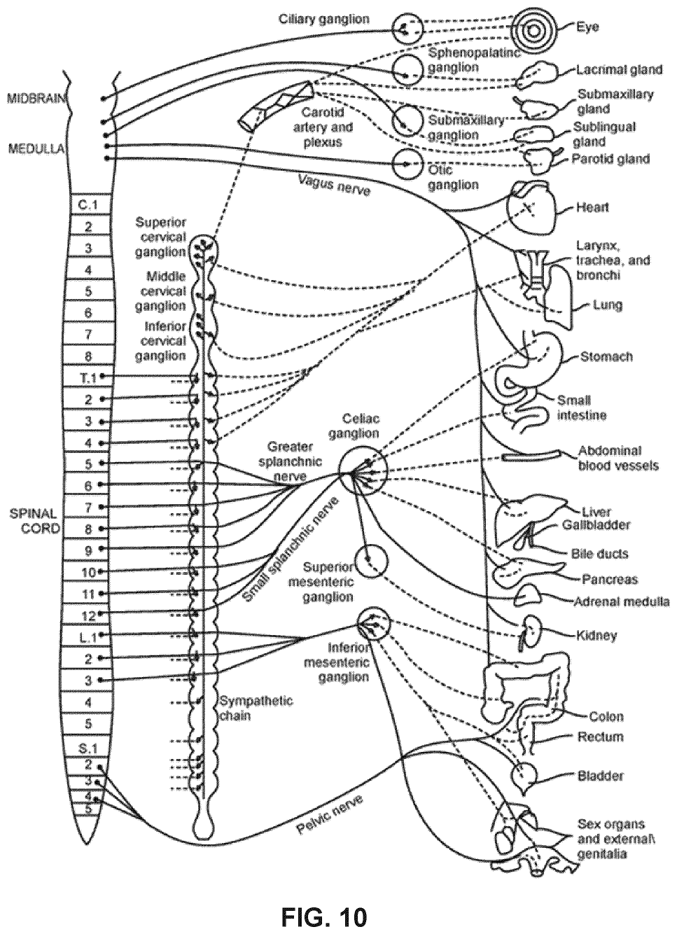

FIG. 10 is a conceptual illustration of an example sympathetic nervous system (SNS) illustrating how the brain communicates with the body via the SNS. As shown in FIG. 10, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of sympathetic nerves leave the spinal cord through the anterior rootlet/root. The axons pass near the spinal (sensory) ganglion, where the axons enter the anterior rami of the spinal nerves. However, unlike somatic innervation, the axons separate out through white rami connectors which connect to either the paravertebral (which lie near the vertebral column) or prevertebral (which lie near the aortic bifurcation) ganglia extending alongside the spinal column.

To reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other component of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cell of the SNS is located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which send sympathetic nerve fibers to the head and thorax organs, and the celiac and mesenteric ganglia, which send sympathetic fibers to the gut.

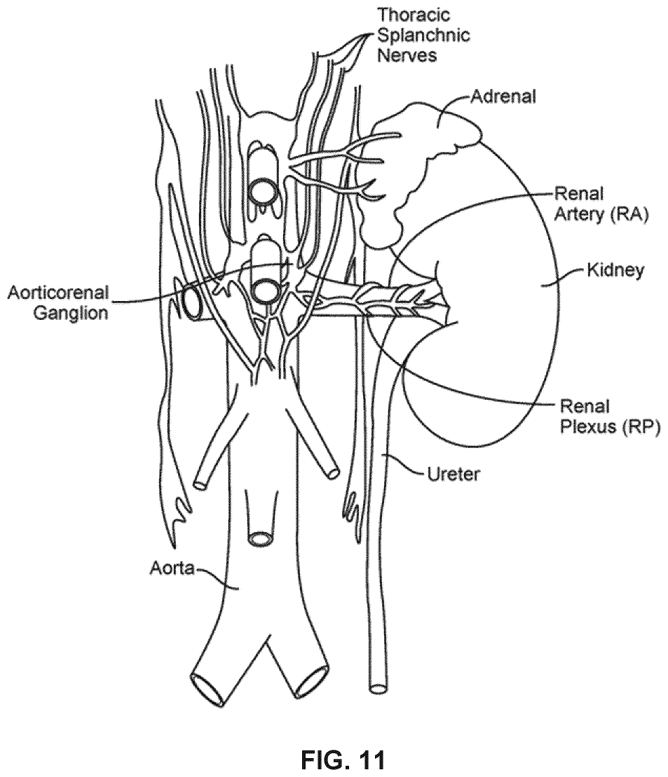

FIG. 11 is an enlarged anatomic view of nerves innervating a left kidney to form the renal plexus surrounding the left renal artery. As FIG. 11 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery and is embedded within the adventitia of the renal artery. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bi-directional flow. Efferent messages may trigger changes in different parts of the body simultaneously. For example, the sympathetic nervous system may accelerate heart rate, widen bronchial passages, decrease motility (movement) of the large intestine, constrict blood vessels, increase peristalsis in the esophagus, cause pupil dilation, piloerection (goose bumps) and perspiration (sweating), or raise blood pressure. Afferent messages carry signals from various organs and sensory receptors in the body to other organs and, particularly, the brain.

Hypertension, heart failure, and chronic kidney disease are a few of the many disease states that result from chronic activation of the SNS, especially the renal sympathetic nervous system. Chronic activation of the SNS is a maladaptive response that drives the progression of theses disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure) and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration late, and left ventricular ejection fraction. These findings support the notion that treatment regimens that are designed to reduce renal sympathetic stimulation have the potential to improve survival in patients with heart failure.

Both chronic and end state renal disease in some patients are characterized by heightened sympathetic nervous activation. In patients with end state renal disease, plasma levels of norepinephrine above the media have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves cause increased renin release, increased sodium (Na⁺) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects, and others.

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As

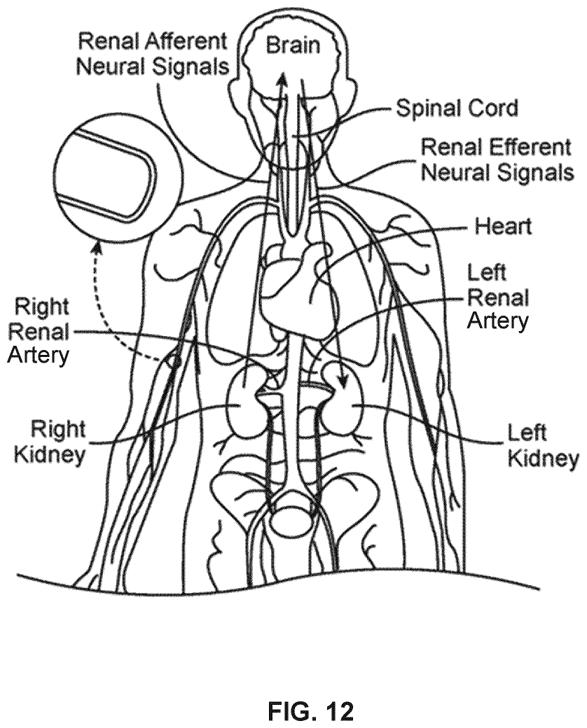

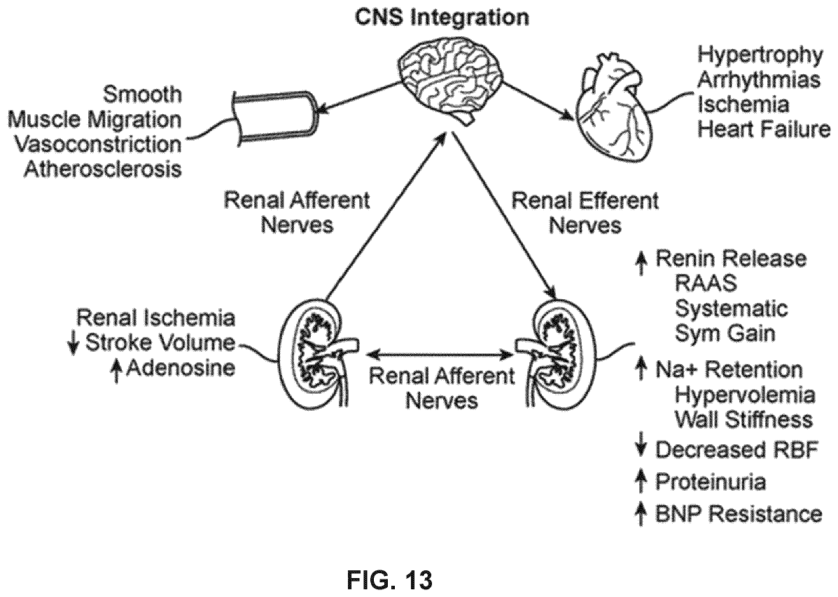

FIG. 12 is an anatomic view of a human body depicting neural efferent and afferent communication between the brain and kidneys. FIG. 13 is a conceptual view of a human body depicting neural efferent and afferent communication between the brain and kidneys. As shown in FIGS. 12 and 13, the afferent communication might be from kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end state renal disease, inappropriate fluid retention in heart failure, cardio-renal syndrome and sudden death. Since the reduction of afferent neural signals contributing to the systemic reduction of sympathetic tone/drive, renal denervation might also be useful in treating other conditions associate with systemic sympathetic hyperactivity. Accordingly, renal denervation may also benefit other organs and bodily structures innervated by sympathetic nerves, including those identified in FIG. 12. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

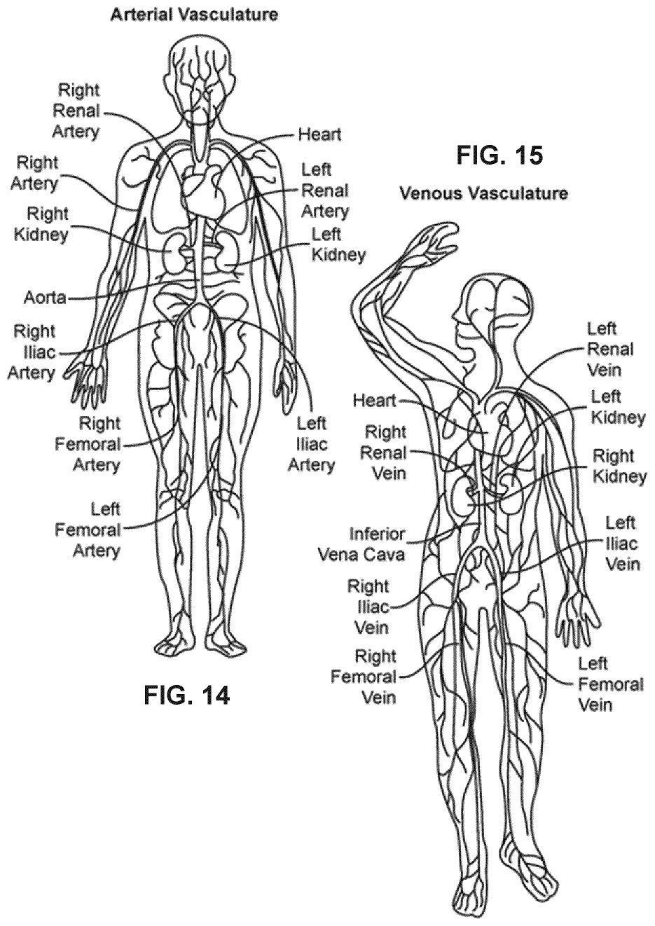

In accordance with the present technology neuromodulation of a left and/or right renal plexus (RP), which is intimately associated with a left and/or right renal artery, may be achieved through intravascular access. FIG. 14 is an anatomic view of the arterial vasculature of a human. As FIG. 14 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

FIG. 15 is an anatomic view of the venous vasculature of a human. As FIG. 15 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

The femoral artery may be accessed and cannulated at the base on the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters (e.g., catheter 102) introduced via these access points may be passed through the subclavian artery on the left side (or via the subclavian and brachiocephalic arteries on the right side), through the aortic arch, down the descending aorta and into the renal arteries using standard angiographic techniques. Other access sites can also by used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

The neuromodulatory apparatus may also be configured to allow for adjustable positioning and repositioning of distal portion 108A and therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that expandable distal portion 108A of catheter 102 may intimately contact the vessel wall and/or extend at least partially through the vessel wall. Renal artery vessel diameter, DRA, typically is in a range of about 2-10 mm, with most of the patient population having a DRA of about 4 mm to about 8 mm and an average of about 6 mm. Renal artery vessel length, LRA, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the artery) to avoid non-target tissue and anatomical structures such as anatomical structures of the digestive system of psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and aorta may vary significantly between patients, and also may vary dynamically within a patient, e.g., due to kidney motion. The take-off angle generally may be in a range of about 30°-135°.

Catheter 102 includes a plurality of filars 302 configured to position one or more therapeutic element 110, such as electrodes, around an inner perimeter of a blood vessel. In some examples in which catheter 102 includes a plurality of therapeutic elements 110, elongated body 108 of catheter 102 is configured and therapeutic elements 110 are positioned along distal portion 108A such that, when distal portion 108A is in the radially expanded helical configuration, therapeutic elements 110 are located at a substantially similar longitudinal position within the vessel. In this way, the neuromodulation catheter may facilitate formation of circumferential lesions around a blood vessel. In other examples, elongated body 108 of catheter 102 is configured and therapeutic elements 110 are positioned along distal portion 108A such that, when distal portion 108A is in the radially expanded helical configuration, therapeutic elements 110 are located at different longitudinal positions within the vessel, which may help distribute the delivery of ablation energy along the longitudinal axis.

Some examples of the disclosure are set forth in the following clauses:
Clause 1: A catheter comprising: an elongated body comprising: a plurality of filars extending axially along the catheter, wherein each filar of the plurality of filars comprises: an outer layer formed from a first material; and an inner core radially inward of the outer layer and formed from a second material that is different from the first material, wherein one of the first material or the second material comprises an electrically conductive material and the other of the first material or the second material comprises a shape memory material; an electrically insulative material configured to electrically insulate at least one filar of the plurality of filars from another filar of the plurality of filars; and one or more electrodes electrically connected to the electrically conductive material of at least one filar of the plurality of filars.
Clause 2: The catheter of clause 1, wherein the plurality of filars is arranged to define a tubular shape.
Clause 3: The catheter of clauses 1 or 2, wherein the first material comprises the electrically conductive material and the second material comprises the shape memory material.
Clause 4: The catheter of clauses 1 or 2, wherein the first material comprises the shape memory material and the second material comprises the electrically conductive material.
Clause 5: The catheter of any of clauses 1-4, wherein the electrically insulative material defines an opening that exposes the electrically conductive material of at least one filar of the plurality of filars, wherein at least one electrode of the one or more electrodes is electrically connected to the electrically conductive material of the at least one filar through the opening.
Clause 6: The catheter of any of clauses 1-4, wherein the electrically insulative material defines a plurality of openings through which the one or more electrodes are electrically connected to the electrically conductive material of the plurality of filars.
Clause 7: The catheter of clause 6, wherein two or more of the electrodes are electrically connected to the electrically conductive material of different filars of the plurality of filars.
Clause 8: The catheter of any of clauses 1-7, wherein each filar of the plurality of filars comprises a layer of the electrically insulative material positioned radially outward of the respective outer layer.
Clause 9: The catheter of clause 8, wherein the layer of electrically insulative material includes a coating of the electrically insulative material over the outer layer.
Clause 10: The catheter of clause 8, wherein the layer of electrically insulative material includes a tubular body formed from the electrically insulative material.
Clause 11: The catheter of any of clauses 1-10, wherein the electrically insulative material fills a space between adjacent filars of the plurality of filars.
Clause 12: The catheter of any of clauses 1-11, wherein the shape memory material comprises nickel titanium.
Clause 13: The catheter of any of clauses 1-12, wherein the electrically conductive material comprises silver.
Clause 14: The catheter of any of clauses 1-13, further comprising a tubular sleeve extending around an outer perimeter of the plurality of filars.
Clause 15: The catheter of clause 14, wherein the tubular sleeve comprises polyethylene terephthalate (PET).
Clause 16: The catheter of any of clauses 1-15, wherein the electrically insulative material comprises a polyether block amide.
Clause 17: The catheter of any of clauses 1-16, wherein the plurality of filars defines an inner lumen, the catheter further comprising an inner liner positioned in the inner lumen.
Clause 18: The catheter of clause 17, wherein the inner liner comprises polyether block amide.
Clause 19: The catheter of any of clauses 1-18, wherein the plurality of filars is configured to expand a therapeutic element radially outwards from a delivery configuration to an expanded configuration.
Clause 20: The catheter of clause 19, wherein in the expanded configuration, the therapeutic element has a maximum cross-sectional dimension of about 1 millimeter (mm) to about 10 mm.
Clause 21: The catheter of clause 19 or clause 20, wherein the plurality of filars defines an inner lumen, configured to receive a guidewire to hold the therapeutic element in the delivery configuration.
Clause 22: The catheter of any of clauses 1-20, wherein each filar of the plurality of filars has a tubular shape.
Clause 23: The catheter of clause 21, wherein the tubular shape defines an inner lumen.
Clause 24: A catheter comprising: an elongated body comprising: a plurality of filars defining a tubular body, wherein each filar of the plurality of filars comprises: a first material; and a second material radially inward of the outer layer and different from the first material, wherein one of the first material or the second material comprises an electrically conductive material and the other of the first material or the second material comprises a shape memory material; an electrically insulative material configured to electrically insulate at least one filar of the plurality of filars from another filar of the plurality of filars, wherein the electrically insulative materials defines one or more openings; and one or more electrodes electrically connected to the electrically conductive material of at least one filar of the plurality of filars through the one or more openings of the electrically insulative material.
Clause 25: The catheter of clause 24, wherein the first material comprises the electrically conductive material and the second material comprises the shape memory material.
Clause 26: The catheter of clause 24, wherein the first material comprises the shape memory material and the second material comprises the electrically conductive material.
Clause 27: The catheter of any of clauses 24-26, wherein two or more of the electrodes are electrically connected to the electrically conductive material of different filars of the plurality of filars.
Clause 28: The catheter of any of clauses 24-27, wherein each filar of the plurality of filars comprises a layer of the electrically insulative material positioned radially outward of the respective outer layer.
Clause 29: The catheter of any of clauses 24-28, wherein the electrically insulative material fills a space between adjacent filars of the plurality of filars.
Clause 30: The catheter of any of clauses 24-29, wherein the shape memory material comprises nickel titanium.
Clause 31: The catheter of any of clauses 24-30, wherein the electrically conductive material comprises silver.
Clause 32: The catheter of any of clauses 24-31, wherein the electrically insulative material comprises a polyether block amide.
Clause 33: The catheter of any of clauses 24-32, wherein the plurality of filars is configured to expand a therapeutic element radially outwards from a delivery configuration to an expanded configuration.
Clause 34: The catheter of clause 33, wherein in the expanded configuration, the therapeutic element has a maximum cross-sectional dimension of about 1 millimeter (mm) to about 10 mm.
Clause 35: The catheter of clauses 33 or 34, wherein the plurality of filars defines an inner lumen configured to receive a guidewire to hold the therapeutic element in the delivery configuration.
Clause 36: The catheter of any of clauses 24-35, wherein each filar of the plurality of filars has a tubular shape.
Clause 37: The catheter of clause 36, wherein the tubular shape defines an inner lumen.
Clause 38: A method comprising: forming one or more openings in an electrically insulative material of an elongated body of a catheter, wherein the electrically insulative material is configured to insulate at least one filar of a plurality of filars of the elongated body from another filar of the plurality of filars, and wherein each filar of the plurality of filars comprises: an outer layer formed from a first material; and an inner core radially inward of the outer layer and formed from a second material that is different from the first material, wherein one of the first material or the second material comprises an electrically conductive material and the other of the first material or the second material comprises a shape memory material; and electrically connecting an electrode to the electrically conductive material of at least one filar of the plurality of filars through the one or more openings.
Clause 39: The method of clause 38, wherein the first material comprises the electrically conductive material and the second material comprises the shape memory material.
Clause 40: The method of clause 38, wherein the first material comprises the shape memory material and the second material comprises the electrically conductive material.
Clause 41: The method of any of clauses 38-40, wherein the shape memory material comprises nickel titanium.
Clause 42: The method of any of clauses 38-41, wherein the electrically conductive material comprises silver.
Clause 43: The method of any of clauses 38-42, wherein each filar of the plurality of filars comprises a layer of the electrically insulative material positioned radially outward of the respective outer layer.
Clause 44: The method of any of clauses 38-43, wherein forming the one or more openings in the electrically insulative material comprises: applying a mask over at least a portion of at least one filar of the plurality of filars; applying the electrically insulative material over the plurality of filars; and removing the mask after applying the electrically insulative material.
Clause 45: The method of any of clauses 38-44, wherein forming the one or more openings in the electrically insulative material comprises using laser etching or mechanical etching to remove a portion of the electrically insulative material.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure.

Certain aspects of the present disclosure described in the context of particular examples may be combined or eliminated in other examples. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within a single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique or any combination thereof.

Moreover, unless the word “or” is expressly limited to mean only a single term exclusive from the other items in reference to a list of two or more items, then the use of “or” in such a list is to be interpreted as including (a) any single item in list, (b) all of the items in the list, or (c) any combination of the items in the list. Additionally, the terms “about” or approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Various aspects of the disclosure have been described. These and other aspects are within the scope of the following claims.

Further disclosed herein is the subject-matter of the following clauses:
1. A catheter comprising:
   an elongated body comprising:
      a plurality of filars extending axially along the catheter, wherein each filar of the plurality of filars comprises:
         an outer layer formed from a first material; and
         an inner core radially inward of the outer layer and formed from a second material that is different from the first material, wherein one of the first material or the second material comprises an electrically conductive material and the other of the first material or the second material comprises a shape memory material;
   an electrically insulative material configured to electrically insulate at least one filar of the plurality of filars from another filar of the plurality of filars; and
   one or more electrodes electrically connected to the electrically conductive material of at least one filar of the plurality of filars.
2. The catheter of clause 1, wherein the plurality of filars is arranged to define a tubular shape.
3. The catheter of clauses 1 or 2, wherein the first material comprises the electrically conductive material and the second material comprises the shape memory material; or wherein the first material comprises the shape memory material and the second material comprises the electrically conductive material.
4. The catheter of any of clauses 1-3, wherein the electrically insulative material defines an opening that exposes the electrically conductive material of at least one filar of the plurality of filars, wherein at least one electrode of the one or more electrodes is electrically connected to the electrically conductive material of the at least one filar through the opening.
5. The catheter of clause 4, wherein two or more of the electrodes are electrically connected to the electrically conductive material of different filars of the plurality of filars.
6. The catheter of any of clauses 1-5, wherein each filar of the plurality of filars comprises a layer of the electrically insulative material positioned radially outward of the respective outer layer.
7. The catheter of any of clauses 1-6, wherein the electrically insulative material fills a space between adjacent filars of the plurality of filars.
8. The catheter of any of clauses 1-7, further comprising a tubular sleeve extending around an outer perimeter of the plurality of filars.
9. The catheter of any of clauses 1-8, wherein the plurality of filars defines an inner lumen, the catheter further comprising an inner liner positioned in the inner lumen.
10. The catheter of any of clauses 1-9, wherein the plurality of filars is configured to expand a therapeutic element radially outwards from a delivery configuration to an expanded configuration.
11. The catheter of clause 10, wherein the plurality of filars defines an inner lumen, and wherein the inner lumen is configured to receive a guidewire configured to maintain the therapeutic element in the delivery configuration.
12. The catheter of any of clauses 1-11, wherein each filar of the plurality of filars has a tubular shape.
13. A method comprising:
   forming one or more openings in an electrically insulative material of an elongated body of a catheter, wherein the electrically insulative material is configured to insulate at least one filar of a plurality of filars of the elongated body from another filar of the plurality of filars, and wherein each filar of the plurality of filars comprises:
      an outer layer formed from a first material; and
      an inner core radially inward of the outer layer and formed from a second material that is different from the first material, wherein one of the first material or the second material comprises an electrically conductive material and the other of the first material or the second material comprises a shape memory material; and
   electrically connecting an electrode to the electrically conductive material of at least one filar of the plurality of filars through the one or more openings.
14. The method of clause 13, wherein forming the one or more openings in the electrically insulative material comprises:
   applying a mask over at least a portion of at least one filar of the plurality of filars;
   applying the electrically insulative material over the plurality of filars; and
   removing the mask after applying the electrically insulative material.
15. The method of clause 13, wherein forming the one or more openings in the electrically insulative material comprises using laser etching or mechanical etching to remove a portion of the electrically insulative material.

1. A catheter comprising:
an elongated body comprising:
a plurality of filars extending axially along the catheter, wherein each filar of the plurality of filars comprises:
an outer layer formed from a first material; and
an inner core radially inward of the outer layer and formed from a second material that is different from the first material, wherein one of the first material or the second material comprises an electrically conductive material and the other of the first material or the second material comprises a shape memory material;
an electrically insulative material configured to electrically insulate at least one filar of the plurality of filars from another filar of the plurality of filars; and
one or more electrodes electrically connected to the electrically conductive material of at least one filar of the plurality of filars.

2. The catheter of claim 1, wherein the plurality of filars is arranged to define a tubular shape.

3. The catheter of claim 1, wherein the first material comprises the electrically conductive material and the second material comprises the shape memory material.

4. The catheter of claim 1, wherein the electrically insulative material defines an opening that exposes the electrically conductive material of at least one filar of the plurality of filars, wherein at least one electrode of the one or more electrodes is electrically connected to the electrically conductive material of the at least one filar through the opening.

5. The catheter of claim 4, wherein the one or more electrodes comprises a plurality of electrodes, and wherein two or more electrodes of the plurality of electrodes are electrically connected to the electrically conductive material of different filars of the plurality of filars.

6. The catheter of claim 1, wherein each filar of the plurality of filars comprises a layer of the electrically insulative material positioned radially outward of the respective outer layer.

7. The catheter of claim 1, wherein the electrically insulative material fills a space between adjacent filars of the plurality of filars.

8. The catheter of claim 1, further comprising a tubular sleeve extending around an outer perimeter of the plurality of filars.

9. The catheter of claim 1, wherein the plurality of filars defines an inner lumen, the catheter further comprising an inner liner positioned in the inner lumen.

10. The catheter of claim 1, wherein the plurality of filars is configured to expand a therapeutic element radially outwards from a delivery configuration to an expanded configuration.

11. The catheter of claim 10, wherein the plurality of filars defines an inner lumen, and wherein the inner lumen is configured to receive a guidewire configured to maintain the therapeutic element in the delivery configuration.

12. The catheter of claim 1, wherein each filar of the plurality of filars has a tubular shape.

13. A method comprising:
forming one or more openings in an electrically insulative material of an elongated body of a catheter, wherein the electrically insulative material is configured to insulate at least one filar of a plurality of filars of the elongated body from another filar of the plurality of filars, and wherein each filar of the plurality of filars comprises:
an outer layer formed from a first material; and
an inner core radially inward of the outer layer and formed from a second material that is different from the first material, wherein one of the first material or the second material comprises an electrically conductive material and the other of the first material or the second material comprises a shape memory material; and
electrically connecting an electrode to the electrically conductive material of at least one filar of the plurality of filars through the one or more openings.

14. The method of claim 13, wherein forming the one or more openings in the electrically insulative material comprises:
applying a mask over at least a portion of at least one filar of the plurality of filars;
applying the electrically insulative material over the plurality of filars; and
removing the mask after applying the electrically insulative material.

15. The method of claim 13, wherein forming the one or more openings in the electrically insulative material comprises using laser etching or mechanical etching to remove a portion of the electrically insulative material.

16. The catheter of claim 1, wherein the first material comprises the shape memory material and the second material comprises the electrically conductive material.

17. The method of claim 13, wherein the first material comprise the electrically conductive material and the second material comprises the shape memory material.

18. The method of claim 13, wherein the first material comprises the shape memory material and the second material comprises the electrically conductive material.

19. A catheter comprising:
an elongated body comprising:
a plurality of filars defining a tubular body, wherein each filar of the plurality of filars comprises:
a first material; and
a second material radially inward of the outer layer and different from the first material, wherein one of the first material or the second material comprises an electrically conductive material and the other of the first material or the second material comprises a shape memory material;
an electrically insulative material configured to electrically insulate at least one filar of the plurality of filars from another filar of the plurality of filars, wherein the electrically insulative materials defines one or more openings; and
one or more electrodes electrically connected to the electrically conductive material of at least one filar of the plurality of filars through the one or more openings of the electrically insulative material.

20. The catheter of claim 19, wherein the one or more electrodes comprises a plurality of electrodes, and wherein two or more electrodes of the plurality of electrodes are electrically connected to the electrically conductive material of different filars of the plurality of filars.
